(19) United States
(12) Patent Application Publication
(10) Pub. No.: US 2009/0104653 A1
(43) Pub. Date: Apr. 23, 2009

(54) Bio-process model predictions from optical loss measurements

(75) Inventors: Barbara Paldus, Woodside, CA (US); Mark Selker, Los Altos Hills, CA (US)

Correspondence Address: Herbert Burkard, BLDG. 1, 3350 Scott Blvd., Santa Clara, CA, 95054, US

(73) Assignee: Finesse Solutions, LLC.

(21) Appl. No.: 11/975,952

(22) Filed: Oct. 23, 2007

(51) Int. Cl. C12Q 1/06

(52) U.S. Cl. 435/39

(57) ABSTRACT

This invention relates to methods for monitoring and controlling bioprocesses. Specifically, it describes using quasi-real-time analytical and numerical techniques to analyze optical loss measurements calibrated to indicate cell viability, whereby it is possible to reveal process changes and/or process events such as feeding or induction. Additionally, the present invention makes it possible to accurately estimate the onset of a decrease in cell viability and/or a suitable time for cell harvesting for a cell culture growth process. Pattern recognition methods for identifying specific process events such as batch feeding, cell infection, and product precipitation are also described.

### FIELD OF THE INVENTION

This invention relates to methods for monitoring and controlling bioprocesses. Specifically, it describes using quasi-real-time analytical and numerical techniques to analyze optical loss measurements calibrated to indicate cell viability, whereby it is possible to reveal process changes and/or process events such as feeding or induction. Additionally, the present invention makes it possible to accurately estimate the onset of a decrease in cell viability and/or a suitable time for cell harvesting for a cell culture growth process. Pattern recognition methods for identifying specific process events such as batch feeding, cell infection, and product precipitation are also described.

### BACKGROUND OF THE INVENTION

Over one third of all drugs now under development by pharmaceutical and biotechnology companies are biotechnology based. Because biological processes involve the synthesis of large and complex molecules such as monoclonal antibodies or recombinant proteins in live cells, more sophisticated manufacturing methods are required to optimize the yield of production runs. Furthermore, the reproducibility and yield of these processes depend on the viability and growth rates of the cells,themselves, their ability to produce the end product, and their stability under varying process conditions.

Today, mammalian cells cultivated in bioreactors have surpassed microbial systems for the production of clinical products, in both product titer and number of products produced. However, significant potential remains to simultaneously increase both the total cell density (TCD), (also called the “packed volume”), and overall viability of mammalian cell cultures, in order to maximize cell mass. Over the past decade, significant improvements have been made in the cell density levels achieved, even in simple batch cultures (See F. M. Wurm, “Production of recombinant protein therapeutic in cultivated mammalian cells”, Nature Biotechnology, 22(11), 1393-8 [2004]). The desire to achieve ever higher cell densities is expected to continue since it is considered to be directly correlated to higher upstream productivities and product yields. Note that a typical mammalian cell density of 10×10⁶ cells/mL and a cell diameter of 10 to 15 micron, the packed cell volume is still only 2 to 3%, whereas some microbial cultures can achieve a packed cell volume of 30% or even more. Moreover, the cell viability in mammalian cell processes tends to be lower than in microbial processes. Typical viability percentages of yeast platforms are in the high nineties, while Escheria Coli processes often produce viabilities in the low to mid nineties. In contrast, many mammalian cell culture processes average only about eighty percent viabilities.

Commonly used mammalian cell lines share metabolic processes and display similar characteristics, such as protein expression, but some cell-line-specific differences can significantly affect performance in production: for example, glycosylation of a given protein can vary across different mammalian systems (See N. Jenkins “Analysis and Manipulation of Recombinant Glycoproteins Manufactured in Mammalian Cell Culture”. Handbook of Industrial Cell Culture: Mammalian, Microbial, and Plant Cells. Vinci V A, Parekh S R, Eds. Humana Press Inc: Totowa, N.J., 2003: 3-20). Suspension cultures are the predominant method of production for mammalian cell cultures used today and typically employ a limited set of cell types, including: Chinese Hamster Ovary (CHO) cells (See L. Chu, D. K. Robinson, “Industrial choices for protein production by large-scale cell culture”, Curr. Opin. Biotechnol. 180-7 [2001]), BHK (B. G. D. Bodecker et al., “Production of recombinant Factor VIII from perfusion cultures: I. Large Scale Fermentation is Animal Cell Technology, Products of Today, Prospects for Tomorrow”, eds. R. E. Spier et al., 580-590, Butterworth-Heinemann, Oxford, U.K. [1994]), HEK-293 (F. M. Wurm and A. R. Bernard, “Large scale transient expression in mammalian cells for recombinant protein production”, Curr. Opin. Biotechnol. 10, 15609 [1999]), SP2/0 (P. W. Sauer et al., “A high yielding, generic fed-batch cell culture for pdocution of recombinant antibodies”, Biotech. & Bioeng. 67, 585-97 [2000]), BALB/c (G. Kohler, C. Milstein “Derivation of specific antibody-producing tissue culture and tumor cell lines by cell fusion”, Eur. J. Immunol., 6(7) 511-9 [1976]) such as mouse myeloma-derived NS0 (L. M. Barnes et al., “Advances in animal cell recombinant protein production: FS-NS0 expression system”, Cytotechnology 32, 109-123 [2000]), human retina-derived pER-C6 (D. Jones et al. “High level expression of recombinant IgG in the human cell line pER-C6”, Biotechnology Prog. 19, 163-8 [2003]) cells, and insect cells, such as Sf9 or Sf21, used in conjunction with the baculovirus expression vector system (BEVS) and (L. Ikonomou et al., “Insect cell culture for industrial production of recombinant proteins”, Appl. Microbiol. Biotechnol. 62(1), 1-20 [2003]).

CHO cells are the most popular cells for mass production of recombinant proteins because of their robustness in suspension, high viability, relatively high packed cell volumes, and compatibility with DHFR and glutamine synthase (GS) based selection for cell line development. SP2/0 and BALB/c cells have an extensive record as a null parent for hybridomas and transfectomas. HEK 293 was found useful in producing recombinant adenovirus and adenoassociated viral vectors (rAAV), and recent developments transient transfection techniques are promoting its use in producing large, glycosylated human proteins. The pER-C6 cell line was originally intended for the production of virus-based products, but has recently been applied to the large-scale manufacturing of a wide range of bioproducts. Insect cell platforms present several advantages over their mammalian counterparts, such as ease of culture, higher tolerance to osmolality and by-product concentration (e.g., lactate), and higher expression levels when infected with a recombinant baculovirus.

Suspension cultures can be implemented in three different types of processes: batch, fed-batch (or extended batch) and perfusion (See Hu, W. S., and Piret, J. M., “Mammalian cell culture processes”, Current opinion in biotechnology 3(2): 110-4, 1992). In batch or fed-batch processes, scale-up to large production volumes is achieved by the successive dilution of a series of bioreactors having increasing volumes. Each smaller bioreactor provides the seed train for the next larger size. Process conditions optimized for a given cell line are usually specific to that line only, and can be characterized by unique process parameters such as glucose consumption, lactate production rate, and sensitivity towards stress signals and/or temperature.

A typical cell growth process has six phases, as shown in FIG. 1. These phases are:
1. Lag phase—zero to minimal cell growth and/or product production; duration depends on how quickly cells adjust to medium, dilution, and new environment after inoculation
2. Accelerated growth phase—cell growth begins and division rate gradually increases to reach the steady state value of the exponential growth phase
3. Exponential growth phase—continued growth of cell population with progressive doubling every division period. Cell density growth is exponential.
4. Decelerated Growth phase—cell population cannot be supported by substrate or waste concentrations in the medium so the growth rate begins to decrease until it reaches zero in the stationary phase.
5. Stationary phase—cell population remains constant because growth rate has been reduced to essentially zero; the cells remain viable but are rapidly exhausting nutrients in the media.
6. Death phase—cells begin to die because the nutrients in the media have been exhausted and/or waste has built up to toxic levels. Similar to cell growth, cell death can become an exponential function. In certain cases, cell not only die, but also disintegrate, so that this phase is sometimes referred to as the “lysis” phase.

The timing of the harvest (termination of the culture) is primarily driven by process kinetics, plant capacity, and desired quality of the derived product. Note that the latter is influenced by the continuously changing composition of the culture medium during the process, such as the build-up of waste products that mediate degradative enzymes, or a dearth of nutrients required to produce the product and/or keep the cells viable. In some processes, harvest begins as early as the decelerated growth phase, while in other processes, additional product is produced in the stationary phase, so that harvest is delayed until the onset of the death phase.

Batch processes are generally the best understood. In a typical batch process (as shown in FIG. 2), the media and cells are placed in a bioreactor, and the reactor runs to completion, whereby the cell population, 11, increases until the substrate, 12, is depleted, and the product production curve, 13, closely mirrors the viable cell density. Typical characteristics of batch processes are:
An isolated system is run under substantially uncontrolled conditions (no data inputs or outputs, minimal control) and has relatively low reproducibility from batch to batch
The initial medium has a substrate (feed) surplus
The run-time (exponential phase) is short
Product is produced only near the end of the run

A fed-batch culture (see FIG. 3a, where 21 is the cell population, 22 is the substrate and 23 is the product) is, in essence, a batch culture which is supplied with either fresh nutrients, growth-limiting substrates, and/or additives, e.g. precursors to products (See Hu, W. S., and Aunins, J. G., “Large-scale mammalian cell culture”, Current opinion in biotechnology 8(2): 148-53, 1997). In fed batch processes, a high concentration of cells is typically first achieved. This is followed by the production of a desired biochemical product induced by the switching of the cell's metabolism from the growth phase to a secondary metabolism. This induction of secondary metabolism may be affected by the depletion of the nutrient required for growth, so that these nutrients may need to be supplemented. Typical characteristics of fed-batch processes are:
The culture starts at less than the full volume of the bioreactor and involves controlled feeding through the addition of fresh medium during the process
Higher biomass and product concentrations than for a batch process
Run-times can be much longer than for a batch process
Product production can occur throughout the process until lysis occurs
Requires on-line analysis for optimization and control

Variations of fed-batch processes include extended and metabolic shift fed batch cultures. In extended fed-batch processes (see FIG. 3b, where 24 is the cell population, 25 is the substrate and 26 is the product), very high product concentrations can be achieved by continuing to feed medium with nutrient concentrates after the cell population has reached a maximum sustainable density. Although the viability of the cells slowly decreases as waste products build-up, the product concentrations can continue to increase substantially. Fed-batch cultures with a metabolic shift (FIG. 3c, where 27 is the cell population, 28 is the substrate concentration and 29 is the product concentration) are initially cultured at low feed substrate concentrations, e.g., low glucose and glutamine concentrations, (See Zhou, W. C., Rehm, J. et al, “High viable cell concentration fed-batch cultures of hybridoma cells through on-line nutrient feeding”, Biotechnology & Bioengineering, 46(6): 579-587, 1995). The growth conditions are controlled so as to maintain the lowest possible substrate concentrations without loss of productivity. The production of protein is often induced by changing the physical or chemical properties of the medium after a sufficiently large cell population exists in the bioreactor. This approach seeks to minimize waste production (e.g., lactate and ammonia) and therefore enhances viability as well as the product titer achieved.

Although most of the commercial systems today use fed-batch approaches, continuously-running perfusion systems are also in use. Perfusion cultures are maintained for several weeks, if not months, with very high cell densities and good cell viability. The media is exchanged several times per day: the old media containing the product is separated from the cells for harvest, and fresh media is continuously added. For example, antihemophilic factor VIII is the largest protein (˜2.3 kd) reliably manufactured using BHK-cells in a perfusion system. Note that this perfusion system, for example, is run on average for up to 6 months at a time. See http://www.pharma.bayer.com/en/products/products/p/productSearchResults.html?country=United+States&product=Kogenate)

The effects of media and feeding on cell viability are poorly understood. Typical goals for feed strategies include replacing depleted nutrients to a cell culture, adding a particular substrate to drive an alternative metabolic pathway, or introducing materials to specifically influence cell apoptosis for harvest. Optimizing media and feed strategies can be difficult, because a culture can increase its cell density ten-fold between the seed phase (original medium) and the feeding time (exponential phase), so that certain components must be brought to significantly higher concentrations.

The simplest feed strategies add concentrated solutions of commercial media or standard amino acids plus glucose and glutamine at mid-culture, i.e., the point in time where the cell density reaches about half of the maximum achievable density (See Huang E P, et al., “Process Development for a Recombinant Chinese Hamster Ovary (CHO) Cell Line Utilizing a Metal-Induced and Amplified Metallothionein Expression System”, Biotechnol. Bioeng. 88(4), 437-450. [2004]). More sophisticated feed strategies add standard media having a high concentration of materials which have been empirically identified as being disproportionately consumed. Even more sophisticated approaches involve influencing or even controlling particular cellular metabolic pathways or activities through controlling the concentration of specific chemical compounds, such as feeding with nucleotide sugars or their precursors to enhance product glycosylation (See Baker K N, et al. “Metabolic Control of Recombinant Protein N-Glycan Processing in NS0 and CHO Cells”, Biotehnol. Bioeng. 73(3), 188-202 [2001]).

In extended fed-batch and perfusion cultures, there is a requirement to maintain high-density cultures for as long as possible in order to produce high yields of end-product, so that cell death (apoptosis) is delayed for as long as possible. Known approaches to controlling apoptosis include adding supplements (apoptosis suppressors) to the medium or supplementing the culture at appropriate times with identified nutritional components, antioxidants, and/or growth factors, as well as maintaining the environmental conditions to be as benign as possible. Conversely, in cases where the harvest phase requires cell lysis, promoters of apoptosis can be added to the cell culture system.

Cell production requires a continued emphasis on bioprocess design and scale-up. The use of process automation and control can help to improve quality, safety, and production costs. Today, the ability to affect intracellular machinery by means of mutant isolation, strain development and genetic manipulation, far exceeds the best available techniques for monitoring/controlling the extracellular parameters in the bioreactor. Specifically, the application of process monitoring and control to biological processes has been limited by the availability of suitable in process, real-time sensors. Many of the key process parameters remain difficult to monitor on-line, and none of them really reflects the real-time changes occurring inside the cells.

Over the past few decades, standardized control procedures have become available for various types of suspension cultures (See J. Lee et al., “Control of Fed-Batch Fermentations”, Biotech. Adv. 17, 29-48 [1999]). Control loops typically operate in either “controlled” or “closed” modes. Closed-loop methods are based on mathematical models, whereas control-loop methods use real-time process measurements and real-time computation of target process settings to feed back to the controlling devices and guide their actions (See B. H. Junker and H. Y. Wang, “Bioprocess Monitoring and Computer Control: Key roots of the Current PAT Initiative”, Biotech. And Bioeng., 95(2), 226-261 [2006]). However, closed-loop methods using simple formulas are not really adequate to accurately describe the evolution of a complex process, so that control-loop methods are usually preferred.

Currently, the basic real-time monitoring instrumentation used in commercial bioreactors only includes dissolved oxygen (DO), pH, temperature, pressure, fluid and foam levels, and optical density (OD). Beyond that, operators must rely on off-line procedures to obtain data on the state of the cells and culture media (both substrates and products). This off-line sampling typically is performed once every four to twenty-four hours. For example, an operator can measure secreted product accumulation using techniques such as ELISA or HPLC or concentration of substrates such as glucose and glutamine using electrochemical methods (See http://www.novabiomedical.com/biotechnology.html). However, cell viability leading to recombinant protein concentration, which is the end-product of interest for most of bioprocesses, or even enzyme activity, have never been effectively monitored on-line and in real time.

In fed-batch processes, real time measurements of DO, pH and cell density could lead to better models or at least better predictive control. Similarly, the ability to make instantaneous glucose and oxygen measurements inside the process vessel would allow the operator to optimize glucose feed rates (timing and quantity) during induction, thereby increasing production yields. The sources of glucose and oxygen must be fed at rates sufficient to maintain the energy needs and viability of the cells for product synthesis, yet not be too high as to cause the cells to switch from production to growth along a more glucose-rich metabolic pathway, and thereby convert glucose to carbon dioxide, which affects the pH and can cause the accumulation of organic acids.

By using feedback control on the feed pump, automatically sampling at periodic intervals from the bioreactor and monitoring the concentration of a nutrient, such as glucose, the feed rate can be optimized to maintain an ideal glucose concentration. The output of the glucose analyzer would ideally be directly tied into the control system as one of many inputs, so that multivariable control is achieved.

A need for more in-line and real-time monitoring is driven by the demand to:
build better mathematical models, feed strategies and control over other operational variables.
produce repeatable, transportable, and operator-independent processes, and
comply with the FDA's process analysis technologies (PAT) initiative.

Because many of the critical parameters cannot be measured in real-time today, it is difficult for the operator to predict how different control strategies will affect cell growth and product production. Many of the existing approaches to process optimization, especially media formulations and feed strategies, therefore remain imprecise, which limits overall productivity of the cell culture system.

New process measurement methods will have an impact on bioprocesses at all scales of operation, from the small amounts required for preclinical studies through to post-license bulk manufacture. Product yields can be increased if monitoring of cell viability is managed properly. Although many factors affect cell growth rates and cell viability, we have found that continuous in-line monitoring of the cell viability can provide a record that the bioreactor environment has been optimized and therefore that the cells will be able to reach their maximum density within a give time frame.

### BRIEF DESCRIPTION OF THE DRAWINGS

FIG. 1 shows the six phases of evolution of the biomass concentration in a typical cell growth (culture) process.

FIG. 2 shows a typical batch production cell culture process

FIGS. 3a, 3b and 3c show the effect of different variations of a fed-batch process: (3a) standard, (3b) extended and (3c) metabolically shifted

FIGS. 4a and 4b show the correlation between (4a) cell concentration and (4b) optical density (OD), and raw turbidity data in absorption units (AU) from a sensor such as is described in U.S. Pat. No. 7,180,594.

FIG. 5a shows the response of a turbidity sensor having a wavelength of 830 nm to an E. Coli bioprocess both in raw AU units and after conversion to cell count (mass). FIG. 5b shows the curve used to convert the raw AU units to cell count.

FIG. 6a shows the response of a turbidity sensor having a wavelength of 830 nm to a CHO cell bioprocess both in raw AU units and after conversion to cell count (mass).

FIG. 6b shows the curve used to convert the raw AU units to cell count.

FIG. 7 shows both cell count and cell viability percentage curves for a mammalian cell culture growth run.

FIG. 8 shows a curve fit of the mammalian cell culture process of FIG. 7 using a Boltzmann function as an approximation to the logistical difference function.

FIG. 9 shows the batch process curve fit and normalized first, 81 and second, 82, derivative functions. Note the feed point, 83, at time t₀=4.923 days, where the cell growth rate reaches a maximum point and the cell growth process becomes limited by the environment (i.e., nutrient availability).

FIG. 10 shows how the extrapolated cell growth curve and its derivative functions as shown in FIG. 9 can be used to predict the onset of the cell growth stationary phase.

FIG. 11 shows a fed-batch process cell growth curve fit and its 1^{st} and 2^{nd} derivatives. In this case, the cell density measurement starts too late in the process to estimate to correctly, so that the prediction of the onset of the stationary phase is incorrect (too early). Note also that the feed step can complicate the process model.

FIG. 12 shows the equation used to solve for the intersection between the normalized first and second derivative curves. The zero cross over points are marked by ovals.

FIGS. 13a and 13b shows a functional fit of cell viability for the processes shown in (13a) FIG. 9 and (13b) FIG. 11. Note that the second crossover point from the first and second derivates was used as the fixed parameter, t_{K}, in the fit.

FIGS. 14a and 14b shows the same functional fit of cell viability for the two processes as FIGS. 13a and 13b, but in this fit, all but the V₁ parameter are fixed and are estimated from the cell density growth curve.

FIG. 15 is a graph showing a fed-batch growth process monitored by a calibrated optical density probe.

FIG. 16 is a graph showing a growth run where the occurrence of a feeding even it not unambiguously indicated by the plotted curve.

FIG. 17 is a graph showing a smoothed version of the graph in FIG. 16 where the first derivative has been taken. The spike clearly reveals the occurrence of the feeding point.

FIG. 18 is a graph of an insect cell growth run where inclusion bodies are formed.

FIG. 19 is shows the growth curve of FIG. 18 after smoothing and the first derivative is taken. The inflection points show the changes in slope where there has been a change in the scattering properties of the cells.

FIG. 20 is a graph showing a plot of the natural log of a typical normalized growth curve vs. time. The slope of the curve is equivalent to the growth rate of the cells.

FIG. 21 shows a series of graphs including a growth curve as detected using a calibrated optical turbidity probe.

FIG. 22 graphically shows the same type of analysis as is illustrated in FIG. 21 but using a different cell line and growth process.

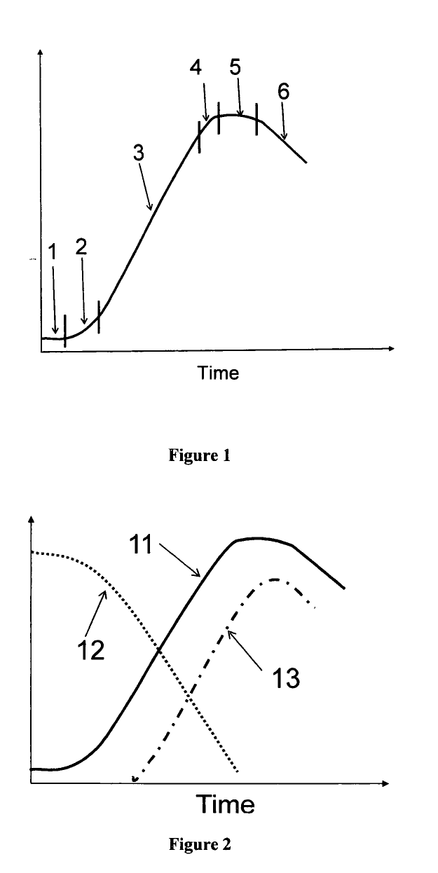

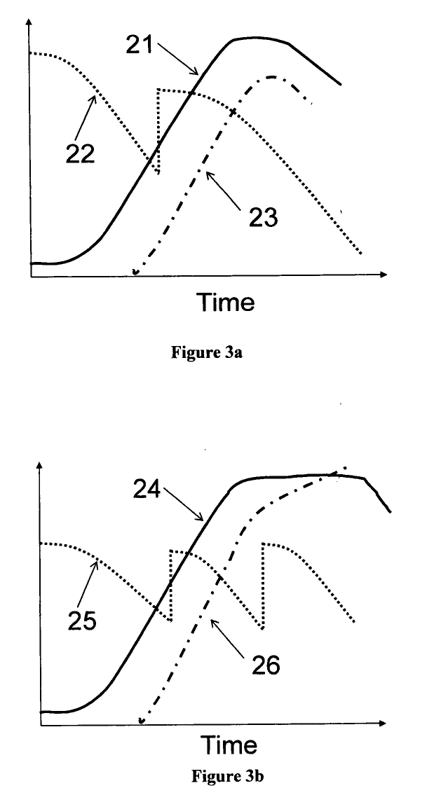

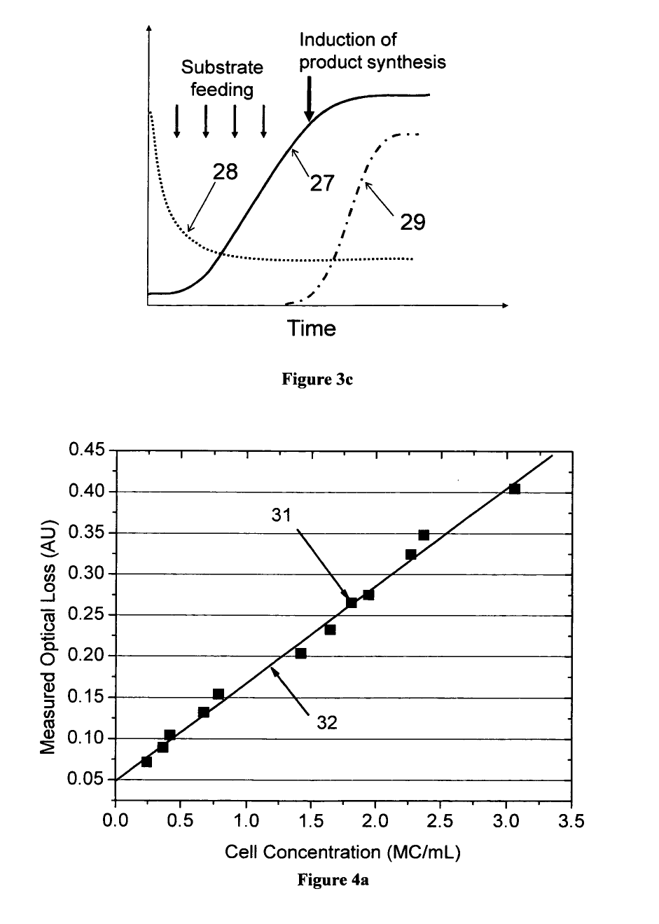

### DESCRIPTION OF THE INVENTION

Monitoring cell growth traditionally has been done with scatter or turbidity type instruments that measure the optical density (OD) generally at visible or near-infrared wavelengths. The cells can be of any variety including but not limited to bacterial, yeast, insect, or mammalian. The only requirement is that the cells scatter the light at the wavelength of the optical source used. Although this approach is generally an indicator of cell density, it has an inherent accuracy problem since it measures the total amount of light both absorbed and also scattered outside the aperture of the optical detector, by all of the living cells, dead cells, cell debris, and in some cases re-absorption by the growth media.

Typical turbidity sensors measure the reduction in transmission of the light (called “optical loss”) as it passes across an optical measurement gap. As the optical loss increases, the amount of the transmitted light decreases. The standard measurement unit of optical loss, Lₒₚₜ, is the absorbance unit (AU). Lₒₚₜ depends on the wavelength, λ, of the light, and is given by equation 1:

$${L}_{opt} ( λ ) = A ( λ ) + S ( λ ) + {L}_{other} ( λ ) = - {log}_{10} ( \frac{{I}_{T} ( λ )}{{I}_{0} ( λ )} ) [ AU ] \qquad eq . 1$$

where: I_{T}(λ)=Light transmitted through sample at wavelength λ
I₀(λ)=Light transmitted through zero/reference solution at wavelength λ
A(λ)=Optical loss through absorption, also called absorbance, at wavelength λ
S(λ)=Optical loss through scattering at wavelength λ, and
   Lₒₜₕₑᵣ(λ)=Optical loss through non-linear effects or measurement processes at wavelength λ.

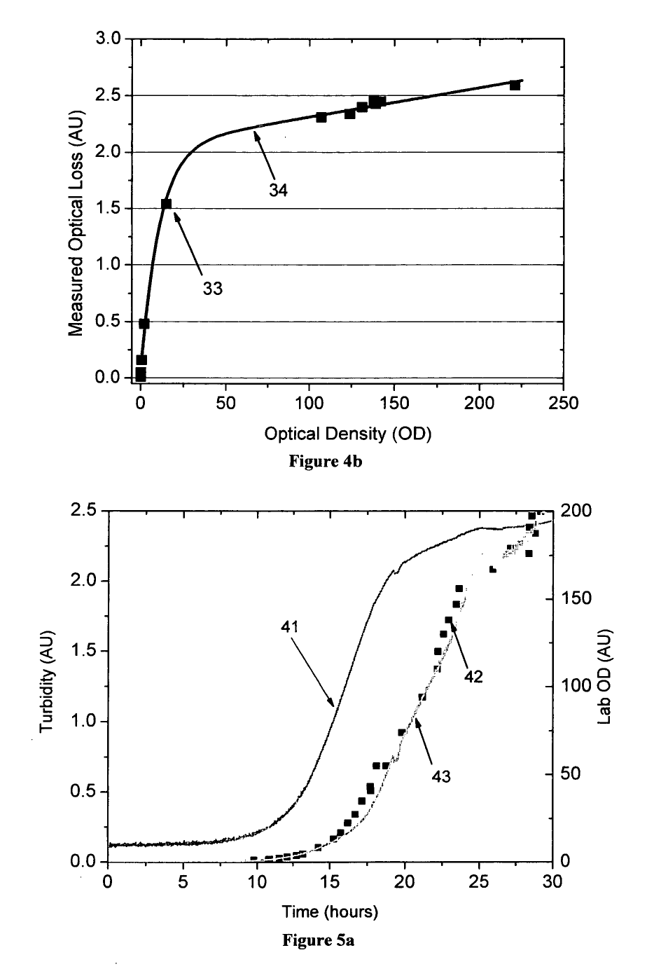

For biological system, the primary optical loss mechanism will frequently be scattering. In cell culture processes where the cell density and scattering losses are relatively low (<1.0 AU), the relationship will be mostly linear (as shown in FIG. 4a). However, in most bacterial fermentation runs where cell densities are high and a higher optical loss is reached, one sees an exponential saturation of the optical loss measurements, owing to significant forward scattering saturating the photodetector response (as shown in FIG. 4b). In FIG. 4a, 31 is the measured data, while 32 is the fit function. In FIG. 4b, 33 is the measured data, while 44 is the fit function.

The critical fact to be born in mind is that in the final analysis what is ultimately of interest is not optical loss, turbidity, or cell density but rather cell viability. The present invention describes and claims processes (and their physical justification in cell population dynamics) that can be used to convert real-time, cell-specific measurements (e.g., cell density, OD, total cell density) into predictable estimates of cell viability and the onset of the death phase, so that a control signal for harvesting can be generated directly from on-line, real-time process data. Also, the present invention describes analytical and mathematical methods for monitoring the occurrence of certain process events, which can be used to predict times for optimal feeding shifts from growth to end product production, or transfection.

One embodiment of the present invention creates a mathematical model which equates measured total cell density and viable cell fraction following an initial calibration based on the viable cell percentage (Vₒ) in the initial cell inoculum. Cell viability can be determined by known methods, such as by using a CEDEX, and is normally close to 100% at the beginning of a run and decreases noticibly as the cell growth process enters the death phase. (See http://www.innovatis.com/products_cedex). This mathematical model enables the bio-process operator to predict the optimal feeding time(s) to maximize cell growth and also to predict the optimal harvest or transfection time by identifying when there is a decline in total cell viability (TCV) greater than a pre-selected standard deviation in the percentage of viable cells.

The mathematical model first provides a curve showing the optically measured total cell density. The curve, if showing undue point scatter can be smoothed using known smoothing algorithms. The first derivative of the natural log of the total cell density yields the specific growth rate (SGR). The knowledge of when the value of the SGR is ˜0 is of value in that it provides the bio-reactor operator with the information necessary to either: i) harvest the cells, ii) take a sample to measure TCV off-line, or iii) realize that an event (good or bad) has occurred in the bio-process such as, for example, the addition of nutrient or undesired change in the pH of the reaction medium.

In order to estimate cell viability from turbidity measurements, a conversion of the turbidity readings into cell density readings must first be made using a fitting algorithm, before a mathematical model can then be applied to convert cell density to cell viability. One such fitting algorithm is described co-pending, commonly assigned U.S. patent application Ser. No. 11/702,861, filed Feb. 6, 2007. Other suitable filtering algorithms are described in the following technical note: (http://www1.dionex.com/en-us/webdocs/4698_{—}4698_TN43.pdf)

The most general fitting function of measured optical loss (turbidity) versus a process parameter such as cell density will typically have the mathematical form:

$${y}_{AU} = A + B ( 1 - {e}^{- \frac{{x}_{PU}}{C}} ) + D \cdot {x}_{PU} \qquad eq . ( 2 )$$

wherein x_{PU} is in the process units (PU), y_{AU} is optical loss in AU, A is the offset, D is the absorption coefficient, B is the effective scattering coefficient, and C is the scattering constant. Such fitting functions are sometimes advantageously utilized for bacterial applications, where the cell concentration can become very high, so that that the scattering loss will tend to dominate.

For mammalian cell culture applications where cell concentrations are frequently low, and the process is sometimes terminated before it reaches the decelerated growth phase, the optical losses measured will be much lower, and a linear approximation will normally be sufficient:

y_{AU}≈A₀=Aₛₗₒₚₑ·x_{PU}

where A₀ is the offset and Aₛₗₒₚₑ≈D+B/C. For mammalian cell cultures that do reach the decelerated growth and stationary phase, because the final product can only be produced once cell reproduction is halted, the process itself, rather than the scattering response of the sensor, saturates. Therefore, equation (2) must be applied with the process units and optical loss variables reversed, namely:

$${x}_{AU} = A + B ( 1 - {e}^{- \frac{{y}_{PU}}{C}} ) + D \cdot {y}_{PU} \qquad eq . ( 3 )$$

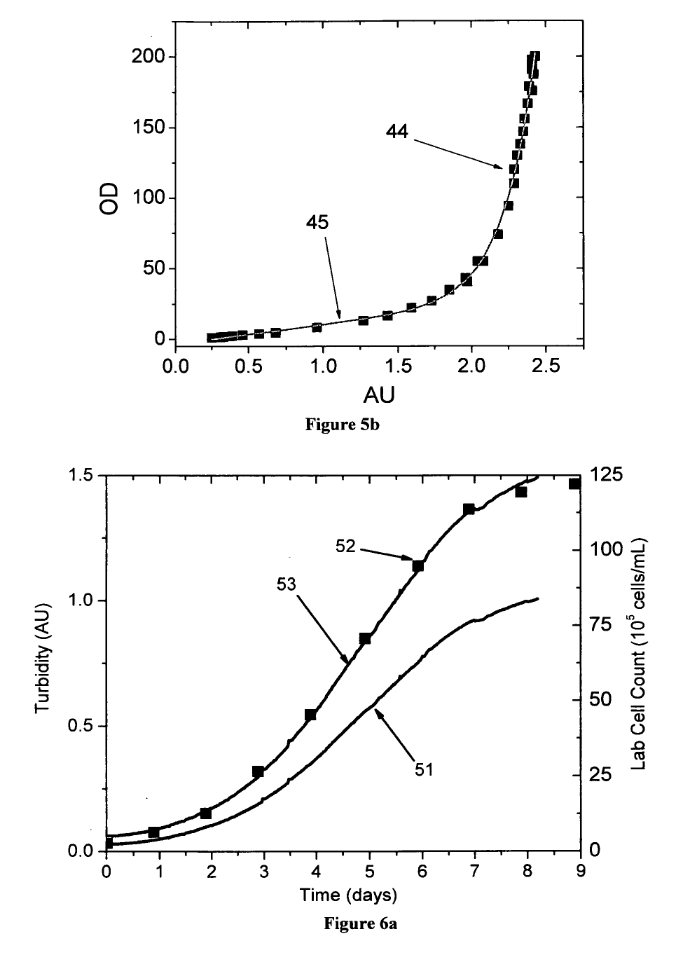

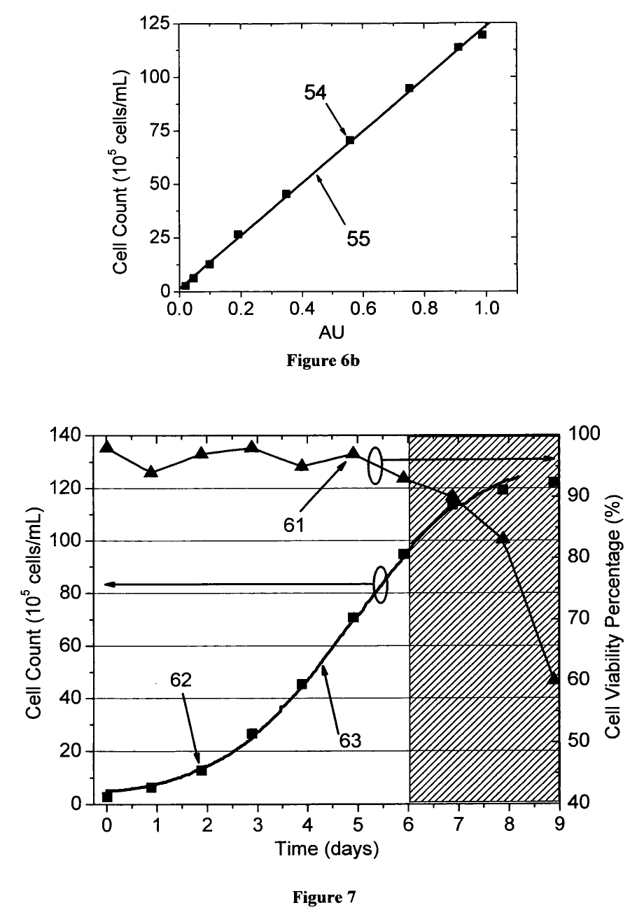

Based on this fitting function, real-time AU measurements by a turbidity sensor can be converted into real-time cell mass or cell density generic functional form describing the evolution of the cell growth process, and mathematical models for cell viability can be derived. FIGS. 4a and 4b illustrates examples of fitting functions used for conversion of turbidity readings in mammalian cell processes (usually linear) or bacterial process (usually non-linear). FIGS. 5 and 6 show a bioprocess growth curve in raw turbidity units (prior to fitting) and in process units (post fitting). In FIG. 5a, 41 is the measured turbidity data in AU, 42 is the measured off-line lab OD data, and 43 is the turbidity data converted into OD units using the fitting function. Similarly in FIG. 6, 51 is the measured turbidity data in AU, 52 is the measured off-line cell count data, and 53 is the turbidity data converted into cell count units.

For mammalian cell culture during the exponential growth phase the cell density typically remains relatively low (<10⁹ cells/mL), and the cell viability remains fairly constant and relatively high (>80%). Therefore, up to the decelerated growth phase, there is a mostly linear correlation between the raw AU measurement and cell density, and the growth process follows traditional growth models, such as is illustrated in FIGS. 6a and 6b. The graph of FIG. 6a shows a typical mammalian cell growth curve, where 51 is the raw AU data, 52 is off-line measured cell count and 53 is the raw AU data converted into cell count units using equation 3. FIG. 6b shows the conversion curve, which is linear where 54 is the actual data, and 55 is the linear fit. FIG. 7 shows the viability percentage, 61, and the correlation between the off-line cell count, 62 and the real-time cell count, 63, based on a conversion from raw AU readings. Note that the viability percentage remains high (>90%) until the decelerated growth phase begins on day 6. At this point, the viability percentage begins to drop more and more rapidly, until the cells enter the stationary phase on day 9.

The observed cell growth curve can be fitted using an analytical equation derived from mathematical models of cell growth and cell volume distributions in mammalian suspension cultures. It is usually assumed that the cells grow in volume to a certain size and then divide in two more or less equal size daughter cells (See G. I. Bell and E. C. Anderson, “Cell Growth and Division I—A Mathematical Model with Applications to Cell Volume Distributions in Mammalian Suspension Cultures”, Biophysical Journal, Vol. 7, p. 329, 1967). The exponential growth is characterized by the “cycle time” or “doubling time” for cell division.

In all of these models, the same differential equation can be used, i.e., the Verhulst-Pearl equation. This equation was first postulated in 1838 by Pierre Francois Verhulst for modeling population growth under the assumptions that the rate of reproduction is proportional to the existing population, and that exponential growth cannot occur forever, because a population is ultimately limited by environmental constraints and resources, so that eventually its growth will slow down to zero. The typical form of this equation is:

$$\frac{d N}{d t} = rN ( \frac{K - N}{K} )$$

Where N is the cell density, K is the bio-reactor carrying capacity, and r is the intrinsic rate of increase of the population. The rate of increase is determined by the cell growth and division models. The carrying capacity is determined by factors such as nutrients and aeration, cell density, and contamination. Note that in most cell culture processes, the carrying capacity is actually time dependent and changes based on the process conditions (which in a bio-process are controlled and can be changed), so that the Verhulst model provides a relatively simplistic view of the actual cell density dynamics. Nonetheless, it can serve as a useful analytical model for harvest prediction and general process control.

The solution to the Verhulst equation is known as the logistic difference function. The typical form of this equation is:

$$N ( t - {t}_{0} ) = \frac{{KN}_{s} {e}^{r ( t - ts )}}{K + {N}_{s} ( {e}^{r ( t - ts )} - 1 )} \qquad eq . ( 4 )$$

where t is time, tₛ is the start time, N is the cell density, N, is the cell density at the start time, K is carrying capacity, and r is the intrinsic rate of increase of the cell population. The initial stage of cell growth is exponential and then as saturation begins, the growth slows (decelerated growth phase) and eventually growth stops (saturation phase). Note that this model does not predict the cell death or lysis phase.

When fitting typical batch cell culture process dynamics, it is often simpler to use a sigmoid curve or Boltzmann curve as an approximation to the logistic difference function. Any of the known approximation techniques to the logistic difference function will normally work equally well. If a fed-batch process is being modeled, a multi-level logistic function may be required for maximum accuracy, because the process conditions are changed by a feed event. In many cases, however, the feed event can be treated as a dilution event (offset) and a single logistic function approximation can again be used. An offset is also usually included in the fit to model the initial population at inoculation. In the examples shown here the following equation was used for data fitting:

$$N ( t ) = {N}_{2} + \frac{{N}_{1} - {N}_{2}}{1 + {e}^{\frac{t - {t}_{0}}{T}}} \qquad eq . ( 5 )$$

Where T is the time constant in the exponential growth phase for t<t₀, t₀ is the time at which the cells move to a linear growth phase which phase is then followed by a decelerated growth phase, N₁ is the cell density at inoculation, and N₂ is the maximum cell density carrying capacity for the process. The cell doubling time, μ, can be computed from the time constant, T, as μ=T·ln2=0.693·T.

The present invention thus provides a process for increasing the cell population at harvest by determining an optimal feeding time or for determining an appropriate time to alter process conditions in order to produce a desired product or to harvest the cells in the course of a second bio-process growth run comprising: i) calibrating an optical turbidity probe to measure cell number density by inserting said probe into the medium in which a first bio-process is being carried out and determining the relationship between the optical loss measured by said probe and the total cell number density obtained by measuring the number of cells present (e.g., by using a CEDEX as previously descriced) in a plurality of bioreactor samples taken over the course of said first bio-process growth run; ii) employing an algorithm to fit the data produced by said calibrated optical turbidity probe during the course of said first bioprocess run to the analytical model of cell number density N at time (t) wherein t denotes a time during the process according to the formula:

$$N ( t ) = {N}_{2} + \frac{{N}_{1} - {N}_{2}}{1 + {e}^{\frac{t - {t}_{0}}{T}}}$$

to thereby determine four parameters of the run, the time constant T, the transition time to when the cells move from the exponential growth phase to the linear growth phase, the initial cell density at inoculation N₁, and the maximum cell density carrying capacity for said bio-process N₂; iii) initiating a second growth run by inoculating a growth medium substantially the same as that utilized in said first growth run with the same cell line as utilized in said first growth run; and iv) adding at least one nutritional additive to said second growth run at time t₀ or changing the physical and/or chemical properties of the medium in said bio-reactor vessel at time t_{H} wherein t_{H}˜t₀+2.71 T.

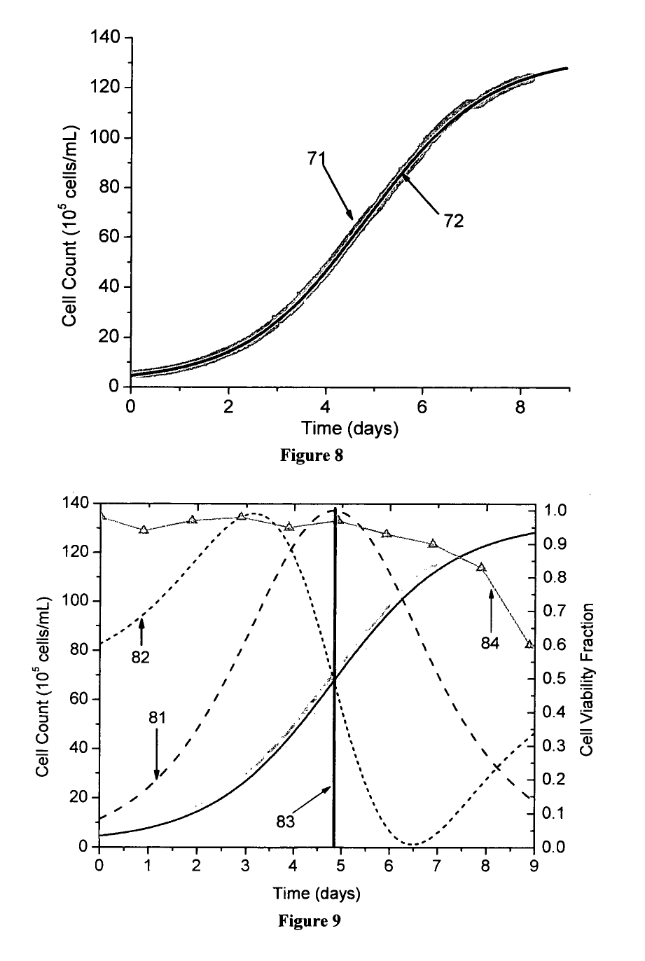

FIG. 8 shows the curve fit of a typical mammalian cell culture process using Equation 5, with good agreement between the measured data, 71, and the Boltzmann fit function, 72. The four fit parameters are: N₁=1.8366×10⁵ cells/mL, N₂=132.997×10⁵ cells/mL, t₀=4.8232 days, and T=1.2617 days. Note that the time constant of the exponential growth phase is 1.26 days and the doubling time is 21 hours. The transition time from exponential growth to decelerated growth occurs at 4.8 days, where the cells have depleted their nutrients. The seed population at inoculation is 2×10⁵ cells/mL, whereas the carrying capacity of the process is about 1.3×10⁶ cells/mL.

The computed first and second derivatives of the Boltzmann curve used to fit the cell culture process are useful in estimating the process phases. The third derivative is used to normalize the second derivative curve. These curves are given by the following equations:

$$\begin{matrix}\frac{d N ( t )}{d t} = \frac{{N}_{2} - {N}_{1}}{T} \frac{{e}^{\frac{t - {t}_{0}}{T}}}{{( 1 + {e}^{\frac{t - {t}_{0}}{T}} )}^{2}} & eq . ( 6 ) \\ \frac{{d}^{2} N ( t )}{d {t}^{2}} = \frac{1}{T} \frac{d N ( t )}{d t} \frac{1 - {e}^{\frac{t - {t}_{0}}{T}}}{1 + {e}^{\frac{t - {t}_{0}}{T}}} & eq . ( 7 ) \\ \frac{{d}^{3} N ( t )}{d {t}^{3}} = \frac{1}{{T}^{2}} \frac{d N ( t )}{d t} \frac{1 - 4 {e}^{\frac{t - {t}_{0}}{T}} + {e}^{{2}^{\frac{t - {t}_{0}}{T}}}}{{( 1 + {e}^{\frac{t - {t}_{0}}{T}} )}^{2}} & eq . ( 8 )\end{matrix}$$

These curves are shown in FIG. 9, where 81 is the first derivative, 82 is the second derivate, and 83 indicates the feed point at t₀. Note that the derivatives have been normalized between zero and one in this example. The first derivative curve is the cell growth rate, whereas the second derivative curve represents the changes in cell growth rate. Note that the first derivative curve reaches its maximum point at t=t₀, i.e., when the second derivative is zero, which is where the cell growth rate is the highest in the process. This point in time marks a feeding point if continuation of the exponential growth phase is desired. Otherwise, the cell growth rate will decelerate, i.e., the first derivative curve begins to decrease beyond this point absent a feed event, as shown in FIG. 9. Therefore, the point t₀ marks the end of the exponential growth phase (absent feeding). Note that up to this point, the cell population environment has been able to maintain exponential growth, so that if the curve fit of the raw AU readings follows the Boltzmann curve, then the cell viability is expected to remain both constant and high. This is indeed the case as shown in FIG. 9, where 84 is the cell viability curve.

For times t>t₀, without feeding, the decelerated growth phase begins. In this phase, the cell growth rate decreases until it reaches zero. Once the growth rate is below the initial growth rate, the cell population reaches the stationary phase and the death phase ensues. In order to estimate the onset of the stationary phase one can solve the equation:

$$\begin{matrix}\begin{matrix}\frac{d N ( t )}{d t} = \frac{d N ( t = 0 )}{d t} \\ = \frac{{N}_{2} - {N}_{1}}{T} \frac{{e}^{{t}_{0} / T}}{{( 1 + {e}^{{t}_{0} / T} )}^{2}} \\ = \frac{{N}_{2} - {N}_{1}}{T} A\end{matrix} & eq . ( 9 ) \\ For x = {e}^{\frac{t - {t}_{0}}{T}} , the analytical solution is \text{:} \\ x = \frac{1}{2 A} ( 1 - 2 A \pm \sqrt{1 - 4 A} ) & eq . ( 10 )\end{matrix}$$

Note that for an “ideal” process, which has a well-defined lag phase, T<<t₀, so that A<<1, and the solutions are x≈A and x≈1/A. The onset of the stationary phase can therefore be estimated as:

$${t}_{i} = T ln ( A ) + {t}_{0} \approx 0 \\ {t}_{s} = T ln ( \frac{1}{A} ) + {t}_{0} \approx 2 {t}_{0} \qquad eq . ( 11 )$$

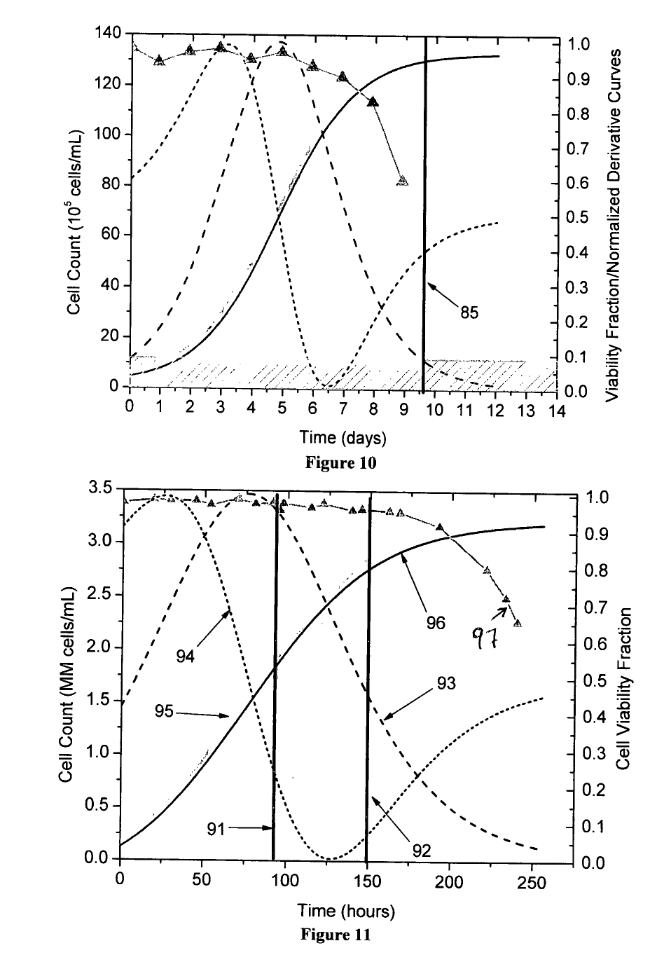

From these equations, a relationship between t₀ and T can found for an “ideal” process, namely that t₀≈T·ln(1/A), which is consistent with the fact that to is the “turning point” in the Boltzmann (or sigmoidal) function, where exponential growth shift from zero shifts to a saturating curve with an exponentially decaying gap. FIG. 10 illustrates how these parameters from FIG. 9 can be used to estimate the onset of the stationary phase, 85, which is 9.65 days after the growth process started. The fit parameters are: A=0.02094, xᵢ=0.02186, xₛ=45.7352, tᵢ=1.1 E-13 and t_{S}=9.646. Note that beyond this point, this mathematical model is not suitable for further predicting the process.

However, not all processes have a well-defined lag phase, because the cell growth measurement can be started too late in the process. In this case, T will be the fixed by the cell line and process (as it is derived from the cell division time) but t₀ will be reduced (because the measurement starts later) and the estimate for the onset of the stationary phase can no longer be used. Furthermore, if there is a feeding point, the process model becomes more complicated, because the exponential growth phase is extended and a single logistic function can no longer be applied. FIG. 11 illustrates these issues in the case of a CHO process where the cell density measurement started after the lag phase (i.e., into the exponential growth phase), and a feed point occurred at 93 hours. The four fit parameters are: N₁=−0.2917×10⁶ cells/mL, N₂=3.224×10⁶ cells/mL, t₀=76.709 hours, and T=38.370 hours. In FIG. 11, 91 is the feed point, 92 shows the onset of the stationary phase, 93 is the first derivative, 94 is the second derivative, 95 is the raw data, 96 is the Boltzmann fit curve and 97 is the cell viability curve. This process did reach a stationary phase where the cell concentration remained constant, and even reached the death phase, where the cell concentration rapidly decreased.

From FIG. 11, we see that the predicted (tₛ˜2t₀) onset of the stationary phase at 153 hours is too early based on the value of t₀. In this example, the Boltzmann curve fit yields t₀˜2·T, so that the above approximations for estimating the stationary phase onset are no longer valid. The process example in FIG. 11 does illustrate, however, that there exists a more useful process control point in the decelerated growth phase, which depends on the process, rather than the curve fit. This point is the cell viability curve “knee”, i.e. the point in time where the cell viability begins to significantly decrease. As we will demonstrate, this point can be empirically estimated using the cross-over point between the normalized first and second derivatives of the cell growth curve, and we have found is independent of cell density measurement start time relative to the cell growth process itself.

The normalization of the first and second derivatives of the cell density curve yields the following functions, for

$$x = {e}^{\frac{t - {t}_{0}}{T}} :$$

:

$$\begin{matrix}{N}_{norm}^{′} ( x ) = \frac{4 x}{{( 1 + x )}^{2}} & eq . ( 12 ) \\ \begin{matrix}{N}_{norm}^{″} ( x ) = \frac{1}{2} + \frac{{( 3 - \sqrt{3} )}^{3}}{2 ( 3 \sqrt{3} - 5 )} \frac{x ( 1 - x )}{{( 1 + x )}^{3}} \\ \approx \frac{1}{2} + 5.1962 \frac{x ( 1 - x )}{{( 1 + x )}^{3}} \\ \approx \frac{1}{2} + 1.299 {N}_{norm}^{′} ( x ) \frac{1 - x}{1 + x}\end{matrix} & eq . ( 13 )\end{matrix}$$

where the maximum and minimum extrema (tₘₐₓ and tₘᵢₙ)of the second derivative are found at:

tₘₐₓ=T ln(2−\sqrt{3})+t₀≈t₀−1.316 T

tₘᵢₙ=T ln(2−\sqrt{3})+t₀≈t₀+1.315 T eq. (14)

Also note that the normalized growth rate is equal (with a value of 0.667) at the two extrema of the second derivative function. One can now proceed to compute the intersection point between the two derivative curves, which will serve as a parameter for the viability curve estimate by solving for x in the equation:

Nₙₒᵣₘ′(x)=Nₙₒᵣₘ″(x). One needs to solve equation 15 for its roots, where

$$B = \frac{{( 3 - \sqrt{3} )}^{3}}{( 3 \sqrt{3} - 5 )} \approx 10.3923 \text{:}$$

x³−(B+5)x²+(B−5)x+1=0

x³−15.3923x²+5.3923x+1=0 eq. (15)

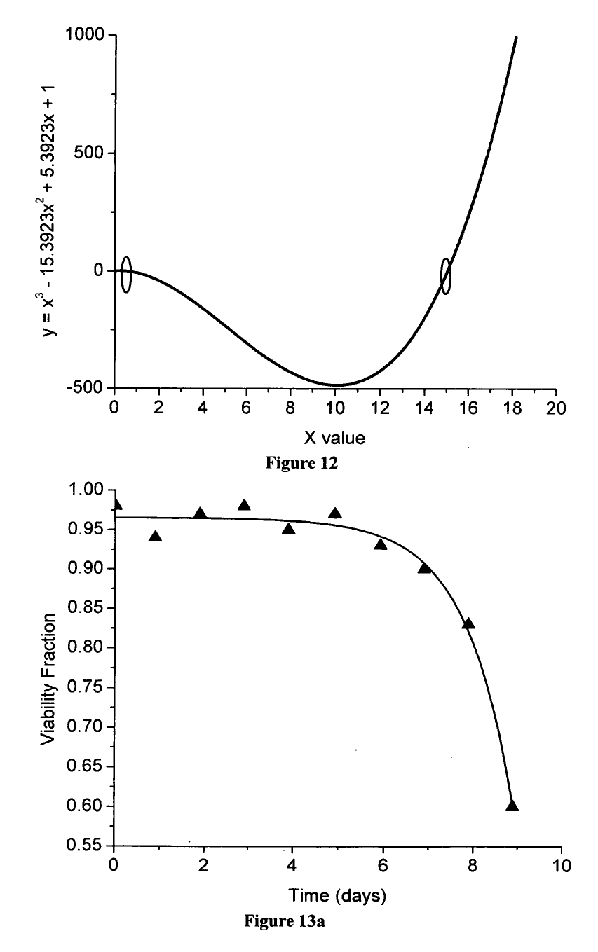

Equation (15) is illustrated in FIG. 12. Using a numerical solver, one obtains positive roots at 0.497 and 15.028, which correspond to process times of

t_{root#1}=T ln(0.497)+t₀≈t₀−0.7 T

t_{root#2}=T ln(15.028)+t₀≈t₀+2.71 T eq. (16)

Using these formulas for the two processes in FIGS. 9 and 11, respectively, we estimate the two curve intersection points to be:

| | Process 1 (FIG. 9) | Process 2 (FIG. 11) |
|---|---|---|
| Left Point | 3.941 days | 49.882 hours |
| Right Point | 8.242 days | 180.688 hours |

This is in agreement with the graphical plots, and demonstrates that irrespective of the mammalian process and the units used, the formulas derived here are suitable to estimate several critical process points, once the curve fit of the cell density curve has been obtained.

The viability curve itself can now be derived. One can assume that the cell viability remains more or less constant during the lag and exponential growth phases, providing the: i) cell density is low enough, and ii) process environment presents the cells with a suitable growth environment having adequate aeration and nutrients, and good temperature and pH control. Once the process reaches the decelerated growth phase, the cell viability begins to decrease until a threshold point is reached, where the process conditions are such that the viability fraction will rapidly decrease. The viability curve can therefore be modeled using the following functional form:

$$V ( t ) = {V}_{0} - {V}_{1} {e}^{\frac{t - {t}_{K}}{{T}_{V}}} \qquad eq . ( 17 )$$

where V₀ is the initial population viability fraction at the beginning of the process, which will be close to 1.0, t_{K} is the time at which the viability fraction begins to exponentially decrease where t_{K}=t_{root#2}, as shown in equation 16. T_{V} is the exponential time constant for the viability decrease, and V₁ is the viability decrease scaling factor which is typically less than 1.0. Note that for

$$t > {t}_{K} + {T}_{V} ln ( \frac{{V}_{0}}{{V}_{1}} ) , V ( t ) < 0 ,$$

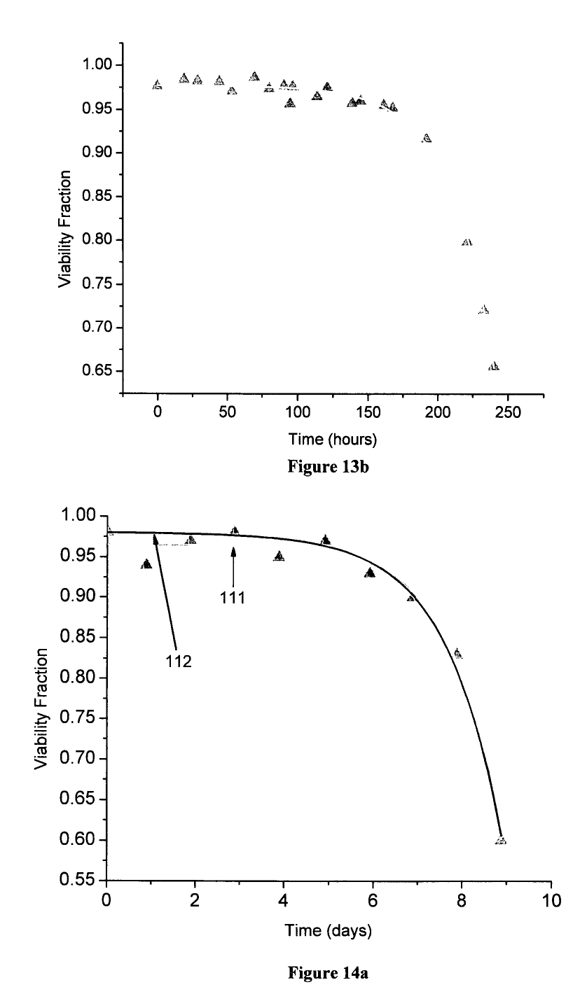

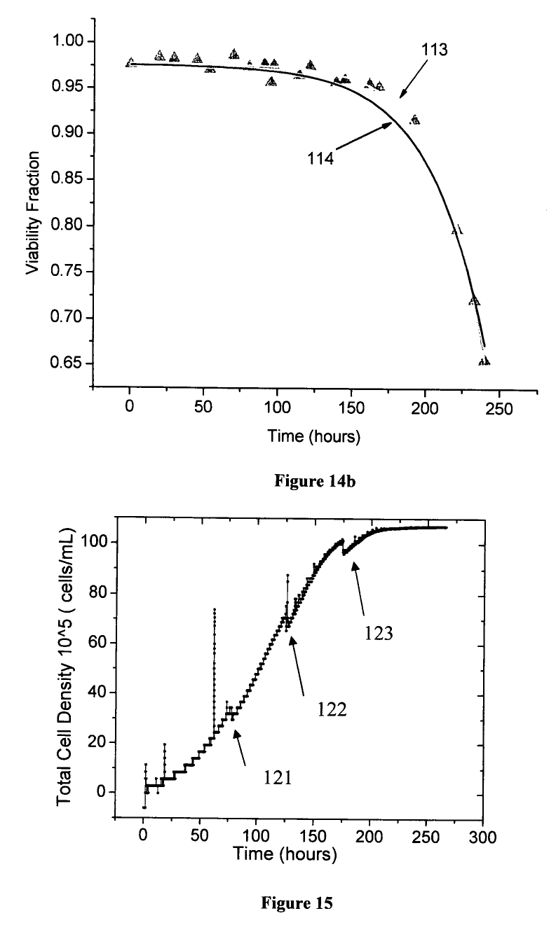

which is no longer physically meaningful. However, as can be seen in FIGS. 13a and 13b and 14a and 14b, which illustrate the curve fits using a fixed t_{K} for the two processes shown in FIGS. 9 and 11, respectively, when V(t) becomes negative, the process time is so far into the stationary/death phases, that the original model is not appropriate. For the process in FIG. 9, the fit parameters are V₀=0.9654, V₁=0.2002, t_{K}=8.242 days, T_{V}=1.0845 days, and V(t)<0 for t>9.95 days. For the process in FIG. 11, the fit parameters are V₀=0.9776, V₁=0.04654, t_{K}=180.67 hours, T_{V}=30.706 hours, and V(t)<0 for t>274.2 hours.

Is it possible to estimate the viability fraction response curve from only the cell density curve parameters by setting the viability time constant to be the same as the growth constant, namely T_{V}=T, and using the viability fraction measurement at the start of the process as the value for V₀. FIGS. 14a and 14b illustrate how, to a first approximation, the curve fits using these estimated parameters are only insignificantly worse than those where all but t_{K} are varied. For the process in FIG. 9, the new fit parameters are V₀=0.9654, V₁=0.2002, t_{K}=8.242 days, T_{V}=1.0845 days, and V(t)<0 for t>9.95 days. For the process in FIG. 11, the new fit parameters are V₀=0.9766, V₁=0.06445, t_{K}=180.69 hours, T_{V}=38.37 hours, and R²=0.9835. The fits with the varying parameters are 111 and 113, while the fits with the all but V₁ fixed are 112 and 114.

Therefore, the cell growth curve parameters can be used to estimate the cell viability curve parameters with only an initial viability measurement of V₀. From a range of studies of different mammalian processes, we have found that the last free variable, V₁, is usually in the range of from about 0 to 0.25 and can be extrapolated using a second viability fraction measurement at the onset of the decelerated growth phase.

Thus, the present invention demonstrates that for fed-batch cell culture processes, which are generally low-noise, it is possible to obtain an analytical model of the cell density curve, from which suitable parameters for feed times and harvest times can be predicted. Similarly, these parameters from the cell density curve can be used to model the cell viability fraction evolution during the bioprocess using the cell density curve parameters as estimates, along with an initial measurement of cell viability. Note that for processes with significant sparging and bubbles, the cell density growth curve noise will advantageously be mathematically filtered out in real-time, or alternatively, numerical methods as will now be described can suitably be employed.

The present invention thus provides a process for determining the percentage of viable cells present in a bio-process medium during the course of a second bio-process growth run comprising: i) calibrating an optical turbidity probe inserted into said medium to measure cell number density by determining the relationship between the optical loss measured by said probe and the total cell number density obtained by measuring the number of cells present in a plurality of bioreactor samples taken over the course of a first bio-process growth run; ii) measuring the cell viability at the onset and end of said first growth run and recording the measurement times of each sample; iii) employing an algorithm to fit the data produced by said calibrated optical turbidity probe during the course of said first bioprocess run to the analytical model of cell number density N at time (t) wherein t denotes a time during the process according to the formula:

$$N ( t ) = {N}_{2} + \frac{{N}_{1} - {N}_{2}}{1 + {e}^{\frac{t - {t}_{0}}{T}}}$$

in order to determine four parameters of the run, the time constant T, the transition time when the cells move from the exponential growth phase to the linear growth phase wherein t denotes a time during the process according to, the cell density at inoculation N₁, and the maximum cell density carrying capacity for the process N₂;iv) initiating a second bio-process growth run by inoculating a growth medium substantially the same as that utilized in said first growth run with the same cell line as was utilized in said first bio-process growth run; v) measuring the initial viable cell fraction (V₀) present in the bioreactor growth medium at the time of initiating said second bio-process growth run; vi) determining the parameters for the cell viability curve in accordance with the equation

$$V ( t ) = {V}_{0} - {V}_{1} {e}^{\frac{t - {t}_{K}}{{T}_{V}}}$$

from the cell growth curve parameters, wherein T_{V}˜T and t_{K}˜t₀+2.71 T, and V₁ is the magnitude of the decrease in cell viability; vii) calculating the percentage of viable cells present in said second bio-process growth run at least once during the course of said second bio-process growth run using the parameters determined in step vi), in conjunction with V₀ as measured in step v); and viii) initiating a change in the bio-process conditions as soon as the percentage of viable cells reaches a pre-determined percentage, based on the calculation of step vii).

Bioprocess automation control systems often utilize a digital computer, a math-coprocessor, or a programmable logic chip. With any of these devices and the appropriate software or firmware it is possible and sometime advantageous to obviate the need for a complete analytical solution to the equations describing cell growth. A computational or numerical analysis often yields information that is difficult to incorporate into an analytical solution. For instance, the cell growth is often affected by a feeding or induction event and a superior analytical solution needs to be able to account for these changes in the cell growth pattern. While these effects can be modeled analytically by breaking the problem into domains, in the end such an approach can end up yielding solutions that are only piecewise continuous. However, a numerical approach as we have developed can be used to create a variety of useful indicators including:
Unambiguous identification that a feeding event has occurred,
Indication of any process change leading to a change in growth rate
Indication of process phases beyond a simple growth process

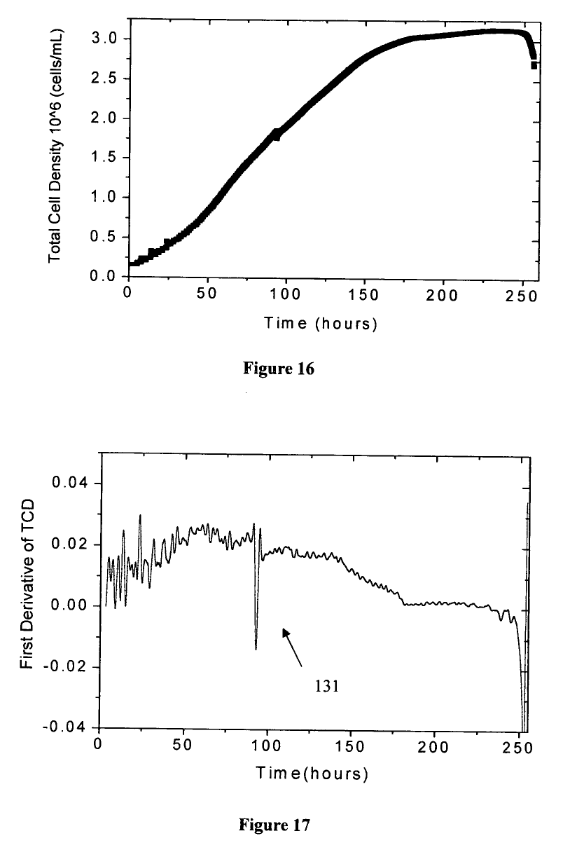

In fed-batch processes the facts that a feeding step has taken place is not always clear from the effect of the feeding on the cell density curve. A typical fed-batch growth process, as monitored using a calibrated optical cell density probe, is shown in FIG. 15. In this figure, the feeding events are clear and marked as 121, 122, and 123. However, when noise from sparging or agitation in the bioreactor is present on the optically derived signal, the feeding events can be difficult to discern. In this case it is often useful to have a filtered or smoothed curve with a numerical derivative to give a clear indicator that the feeding event has taken place. FIG. 16 shows a growth run where afeeding event is not unambiguously clear. FIG. 17 shows the curve that results after numerical signal processing has occurred, including using Savitzky-Golay smoothing (See A. Savitzky and Marcel J. E. Golay (1964). Smoothing and Differentiation of data by Simplified Least Squares Procedures. Analytical Chemistry, 36: 1627-1639) and then numerically taking the first derivative. In FIG. 17 when a sharp change in the slope of the growth curve in FIG. 16 occurs, it is indicated by peak 131 in the first derivative at about 95 hours into the growth cycle. This peak can be used by the bio-process run operator to confirm that a feeding event has occurred. Absent this information, an operator would be unaware or at least uncertain that a system or component malfunction had occurred and that a desired or even essential feeding had (or had not) not taken place. Additionally, a threshold can be set by which peaks like this are counted and the occurrence of such peaks automatically correlated to the feeding command. This allows the system to send a message to the user and to confirm in the data logging system that the desired action has been completed.

Since the response of an optical cell density probe is determined by the scattering properties of what is present in its optical gap, it can respond to changes in cell structure or make-up as well as cell density. The change in response is due to the change in scattering properties of the cells as physical changes occur. This is often of interest during a bioprocess as these changes are often purposely induced. For example, when the cells are in, or have passed, the exponential growth phase, the temperature is often changed or an enzyme is added to induce the cells to create a desired product. The product can be secreted by the cell or form a solid within the cell (often referred to as an inclusion body). When the cells form inclusion bodies, the optical scattering properties of the cells frequently change noticeably. As previously mentioned, this change is picked up by an optical cell density probe despite the fact that there has not been a change in cell number density. If these changes are noted, they can often be correlated to a change in the cells by off-line examination with a microscope, or by testing for a chemical change in the supernatant liquid. If the bioprocess is well characterized and well controlled, the change in the optical signal or the slope of the optical signal can be used as an indicator of the process. This obviates the need to perform costly offline examinations and to break the sterile barrier of the bioreactor.

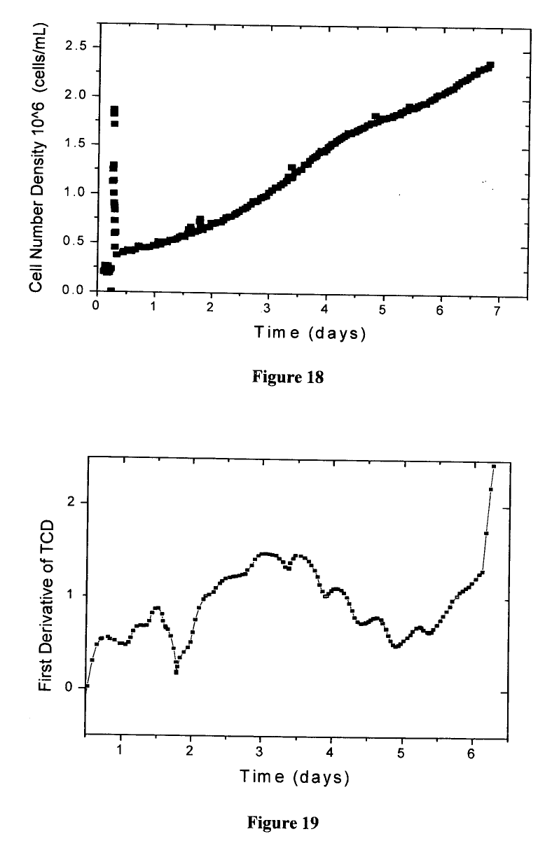

As an example, FIG. 18 shows a growth run which is typical of a bioprocess using insect cells where an inclusion body is formed. The cells are infected with Baculovirus during the exponential growth phase and then begin to form polyhedral crystals as inclusion bodies. The optically detected TCD curve in FIG. 18 shows some subtle changes in slope that are indicative of process changes in the cells. These changes are reflected by large changes in the instantaneous slope, or first derivative, of the growth curve. This instantaneous derivative is shown in FIG. 19. It can be readily seen that the slope changes in FIG. 18 are contemporaneous with the dips in the slope of the curve in FIG. 19 at approximately the 2 hour point and the 5 hour point. Though the overall curve cell density values have been correlated to offline measurements, these changes in the slope indicate of a change in the scattering properties of the insect cells due to inclusion body size changes and not due to cell growth.

A numerical approach in accordance with the present invention can also be used to generate an indicator of cell viability from the measured cell density. Before describing the numerical techniques suitable to generate this indicator, it is helpful to understand what affects the ability to accurately and repetitively derive a useful signal that indicates a change in the total cell viability (TCV) from a total cell density (TCD) measurement. These factors include:
The ability of the probe measuring the TCD to give a signal that is truly proportional to TCD,
The required time response of the system,
The availability of calibration data for both TCD and TCV

As discussed before, the signal coming from one type of known optical cell density probe (see e.g., U.S. Pat. No. 7,180,594,) is proportional to the optical loss across a gap. The optical probe is inserted into the bioreactor and the probe gap is thereby filled with the growth media under study. The optical loss generated by traversing the gap is generally proportional to the mean TCD, but can manifest variations due to a variety of effects. These effects include but are not limited to the break-up of cells and the concomitant scattering debris, and/or the cell-internal production of inclusion bodies. These effects lead to a change in the effective index of refraction of the cell and hence its scattering properties. These changes in the scattering properties change the AU reading that the probe records. As mentioned before, it is then possible to have a scenario where the cell number density does not change and yet scattering properties do change. Additional complications can be envisioned where cell lysis has occurred and the cell is starting to break apart. In this scenario, these cells should no longer be counted in the TCD and yet they still contribute to the overall scattering function of the media. As before, this scattering can lead to deviations from the idealized mode. Most of these issues are, however, overcome by creating a mapping of the optical loss values to TCD values generated by known prior art cell counting methods. (e.g.: Trypan Blue Method http.://www.bio.com/protocolstools/protocol.jhtml?id=p2151) By characterization of the growth curve and correlation of the scattering function and its derivative to offline sampling, all of the apparent anomalies can be used as markers for the bio-process under study.

The next issue which it is appropriate to address is the contraction and delay of the data generation that occurs when using smoothing and averaging techniques required in grooming the data stream and reducing spurious noise. Data smoothing and averaging and even numerical derivatives can sometimes require several samples or even tens of samples to have the desired effect. For instance, a running average will truncate the data stream proportionally to the number of samples averaged. Specifically, if the moving average is taken over n data points, the list of points will be shortened by n−1 points. If the data set is 200 points long and it is averaged with a 25 point moving average, the resulting averaged data set will be 176 points long. These same mathematical operations on the data points then can also lead to a delay in getting the numerical signal by n−1 data points, if using averaging. Smoothing techniques often use data points both before and after the point being processed, so the delay will depend on the which algorithm (a simple averaging scheme or Savitzky-Golay smoothing or another) is used and how many data points in each direction are involved. Assuming symmetric smoothing and that the data is sampled once every minute, this translates to a delay of n−1 minutes. The actual amount of averaging or filtering required is directly dependent on how noisy the data is and therefore how much filtering and smoothing is required to get a usable signal data set. The delay also depends on the frequency of sampling, which will, in turn, depend on the details of the bioprocess, the data acquisition system, and the particular algorithms implemented. For many mammalian systems where growth runs are typically between 7 and 21 days, time delays of even tens of minutes are not an issue. Additionally, this numerical indicator can also be used to simply prompt the user to employ an off-line method to unambiguously determine the parameters of interest. However, for a bacterial bioprocess using e.g., Escheria-Coli, a full run can be less than 72 hours and therefore time delays can sometimes be more important. In general, however, the sampling time will scale with the time it takes to perform the growth run, and as the delay is generally related to the number of samples that are required, all issues will scale accordingly.

Another issue to be addressed is the ability to calibrate the calculated values. As mentioned previously, true cell density measurements are often mapped on the readings of optical turbidity probes in order to enable the probes to read out units and values that are directly relevant to the process being monitored. With the numerical technique of the present invention it is necessary to correlate the changes in cell density values to the onset of changes in cell viability through a similar process.

The basic equations for either mammalian or bacterial cell growth involve an exponential or sigmoidal behavior. Following Zwietering (See M. H. Zwietering et al., Modeling of the Bacterial Growth Curve, Applied and Environmental Microbiology, June 1990, p. 1875-1881) one can take a simplified view of the equations describing cell growth. Zwietering reviewed various models of cell growth and used a general analysis to critique the overall inconsistencies in the terminology used by various authors. He assumed that bacteria grow exponentially and therefore that by examining the function, y, the natural logarithm of the normalized growth curve, it would be possible to plot a quantity proportional to that exponent. The mathematics is shown below:

y=ln(N/N₀)

where:
N=cell number density
N₀=initial cell number density

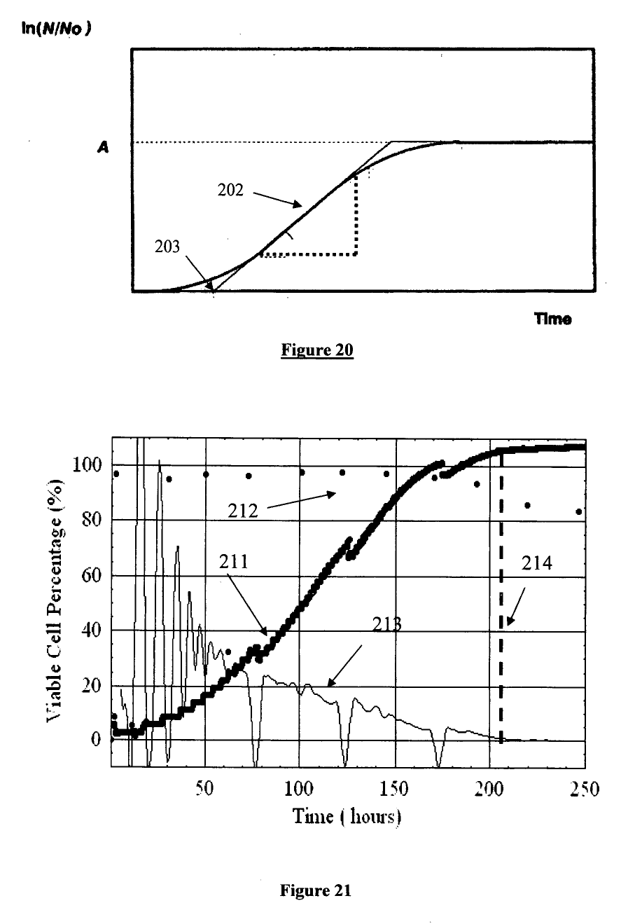

A graph of the generalized model is shown in FIG. 20 based on Zwietering's paper. In this figure the natural log of the normalized growth curve is plotted vs. time. In FIG. 20, (202) is the quantity μₘ (203) which is often referred to as the specific growth rate and is given by the slope of the curve, while the quantity λ is the point in time at which the growth curve would initiate if the specific growth rate were a constant. This term is used because the mathematical value of the exponent yields the rate of change in the growth process The issue noted by Zwietering is that it is necessary to decide over what range the curve is linear in order to make this fit and what the start point λ is. By taking the natural log of an exponential curve one retrieves the exponent μₘ which, as noted above, is called the specific growth rate. However, as we have described, the cell growth is not always exponential and it does not always have the same specific growth rate, and as can be seen in FIG. 20, the curve is not a straight line. At the beginning of the growth curve the rate is not exponential, and likewise at the end, in the death phase, the curve is not exponential. This is one reason that we referred to the Verhulst equation earlier in our analytical model, as most population curves can be fit well by sigmoidal functions. However, by taking the first derivative of the natural logarithm of the growth curve, it is possible to see the “instantaneous” change in what corresponds to a specific growth rate at every point in time. Additionally, if we define cell viability as the ability of the cell to grow and reproduce, then when the specific growth rate goes to zero, the cell viability has likewise tended towards zero. While the definitions of specific growth rate and cell viability are distinct and different, they are related. We show that this calculation serves as a valid and correlated indicator of cell viability as measured with standard off-line measurements.

The present invention thus provides a process for determining changes in the instantaneous specific growth rate of cells in a bio-process comprising the steps of: i) inoculating a growth medium contained in a bio-reactor vessel with cells; ii) plotting a first curve using a calibrated optical turbidity probe, which first curve plots the number density of said inoculated cells vs. time; iii) smoothing the data from said first curve using a Savitzky Golay smoothing algorithm; iv) calculating the first derivative of the smoothed curve to thereby provide a second curve indicative of the specific growth rate of said cells relative to the time elapsed since inoculation; v) determining any discontinuities in said second curve; and vi) recording the time at which said discontinuities occur relative to the time elapsed since said inoculation, or determining from said second curve when the specific growth rate decreases to substantially zero.

FIG. 21 shows a series of curves including a growth curve as detected using a calibrated optical turbidity probe. In FIG. 21, (211) is the TCD curve shown in FIG. 16a, with limited numerical processing done. In FIG. 21 a filter has been employed that removes a point if it exceeds its predecessor by more than a factor of 1.5; additionally, the curve has been scaled to fit in the graph, which is appropriate for cell viability. In FIG. 21 the other two curves are the viable cell percentage (212) and our numerically derived curve (213). The curve labeled number 212 is the actual cell viability determined using a CEDEX (See http://www.innovatis.com/products_cedex) and shows the typical trend where the TCV is close to 100% at the beginning of the run and decreases noticibly as the cell growth process enters the death phase. The curve labeled number 213 in FIG. 21 is what we refer to as an instantaneous specific growth rate and which was calculated as previously discussed. The calibrated cell density data was smoothed with a Savitzky-Golay smoothing filter that used a second order polynomial and 8 points leading and 8 points trailing the point to be smoothed. The natural log of the data was then taken and then smoothed again with a second order Savitzy-Golay filter using 5 points leading and 5 trailing. Finally, the numerical derivative was taken and a 3 point running average on the data was taken and the data scaled to fit in the cell viability graph. The initial spikes in the data before 50 hours are due to the fact that the data was under-sampled on the data logger. This under-sampling has resulted in a “stair-case” stepping of the data as is clear in FIG. 21. The derivative of this stepping behavior creates physically meaningless large spikes in the calculated growth rate in response to the clear discontinuities between steps. However, as shown previously, a discontinuity can be physically meaningful in the case of feeding events. As before, with a simple derivative of the TCD curve the large discontinuities due the dilution during feeding result in clear spikes in the derivative which can be used in conjunction with a threshold to indicate completion of a feeding event. Also shown in FIG. 21 is the temporal correlation between the decay in the viable cell percentage (as measured using offline methods) and the instantaneous specific growth rate going to zero. This correlation in time is marked by the vertical line labeled as 214. As noted before, if the cell viability decreases markedly their ability to grow, divide, and/or produce product is impaired. Therefore, it is possible to use the instantaneous growth rate as an indicator of cell viability and therefore an indicator of the appropriate harvest time for the cells.

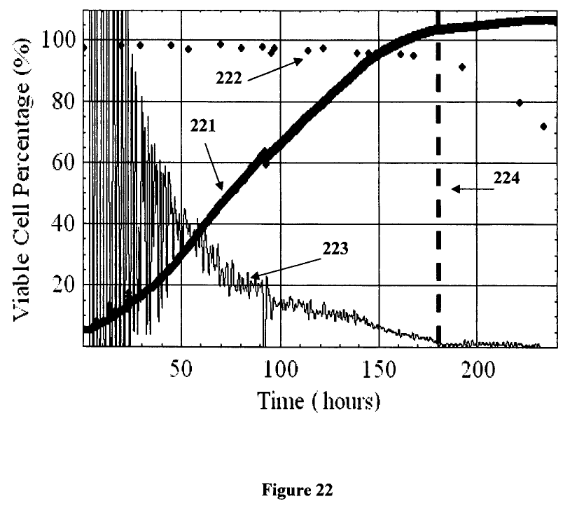

In order to further verify this hypothesis, an identical analysis on a different cell line and growth process was performed. In FIG. 22 the curve labeled as 221 is the total cell density curve of FIG. 16 shown after similar initial data conditioning. As before, an algorithm was employed to remove the physically non-relevant peaks in the data which were likely caused by bubbles passing through the optical gap of the turbidity probe and causing large deviations from the true optical loss of the sample. Similarly to before, a second order Savitzky-Golay smoothing algorithm was employed both before and after the natural logarithm of the amplitude was cancelled. The numerical derivative was performed and the data was subsequently smoothed and averaged. In FIG. 22, 221 is the optically recorded and calibrated total cell density curve scaled to fit in the window; 222 is the actual cell viability taken off-line with a CEDEX as in FIG. 21. This curve sets the scale for the graph, 223 is the numerically derived indicator just discussed; and as before, it has been scaled to fit in this graph. Finally, 224 is a line showing the correlation between calculated instantaneous growth going to zero, and the change in the measured cell viability. As in FIGS. 16a and 16b, the correlation between the feeding events and the spike in the first derivative is clear. Although there is still noise on the instantaneous specific growth rate curve in the first 50 hours due to the discrete steps in the data, but these can be averaged out if desired.

1. A process for increasing the cell population at harvest in a subsequent bio-process growth run by determining an optimal feeding time for said subsequent growth run comprising:
i) calibrating an optical turbidity probe to measure cell number density by inserting said probe into the medium in which a first bio-process is being carried out and determining the relationship between the optical loss measured by said probe and the total cell number density by measuring the number of cells present in a plurality of bioreactor samples taken over the course of said first bio-process growth run;
ii) employing an algorithm to fit the data produced by said calibrated optical turbidity probe during the course of said first bioprocess run to the analytical model of cell number density N at time (t) wherein t denotes a time during the process according to the formula:
$$N ( t ) = {N}_{2} + \frac{{N}_{1} - {N}_{2}}{1 + {e}^{\frac{t - {t}_{0}}{T}}}$$
to thereby determine four parameters of the run, the time constant T, the transition time t₀ when the cells move from the exponential growth phase to the linear growth phase, the initial cell density at inoculation N₁, and the maximum cell density carrying capacity for said bio-process N₂;
iii) initiating a subsequent growth run by inoculating a growth medium substantially the same as that utilized in said first growth run with the same cell line as utilized in said first growth run; and
iv) adding at least one nutritional additive to said subsequent growth run at time t₀.

2. The process in claim 1, further comprising the step of adding additional media to the bioreactor at time t₀.

3. A method for determining an appropriate time to alter process conditions in the course of any subsequent bio-process growth run in order to produce a desired product or to harvest the cells produced by said subsequent bio-process during the course of said subsequent bio-process growth run, said method comprising:
i) calibrating an optical turbidity probe to measure cell number density by inserting said probe into the medium in which a first bio-process growth run is being carried out and determining the relationship between the optical loss measured by said probe and the total cell number density obtained by measuring the number of cells present in a plurality of bioreactor samples taken over the course of said first bio-process growth run;
ii) employing an algorithm to fit the data produced by said calibrated optical turbidity probe during the course of said first bioprocess run to the analytical model of cell number density N at time (t) wherein t denotes a time during the process according to the formula:
$$N ( t ) = {N}_{2} + \frac{{N}_{1} - {N}_{2}}{1 + {e}^{\frac{t - {t}_{0}}{T}}}$$
to thereby determine four parameters of the run, the time constant T, the transition time when the cells move from the exponential growth phase to the linear growth phase t₀, the cell density at inoculation N₁, and the maximum cell density carrying capacity of said process N₂;
iii) initiating a subsequent growth run by inoculating a growth medium substantially the same as that utilized in said first growth run with the same cell line as utilized in said first growth run; and
iv) changing the physical and/or chemical properties of the medium in said bio-reactor vessel at time t_{H} wherein t_{H}˜t₀+2.71 T.

4. The method of claim 3, wherein at time t_{H} the cells are harvested or caused to produce a selected protein, enzyme, viral vector, or antibody product.

5. The method of claim 3, wherein the cell culture process temperature and/or pH is changed at time t_{H}.

6. The method of claim 3, wherein the nutrient concentration is increased in the bioreactor vessel at time t_{H}.

7. The method of claim 4, wherein the cells are harvested at time t_{H}.

8. The method of claim 3, wherein the cells are transfected with an adenovirus or baculovirus at time t_{H}.

9. A process for determining the percentage of viable cells present in a bio-process medium during the course of a subsequent bio-process growth run comprising:
i. calibrating an optical turbidity probe inserted into said medium to measure cell number density by determining the relationship between the optical loss measured by said probe and the total cell number density obtained by measuring the number of cells present in a plurality of bioreactor samples taken over the course of a first bio-process growth run;
ii. measuring the cell viability at the onset and end of said first growth run and recording the measurement times of each sample;
iii. determining the parameters for the cell viability curve in accordance with the equation
$$V ( t ) = {V}_{0} - {V}_{1} {e}^{\frac{t - {t}_{K}}{{T}_{V}}}$$
where V₀ is the viability at inoculation, t_{K} is the time at which viability begins to decrease, T_{V} is the time constant of the decrease, and V₁ indicates the magnitude of the viability decrease;
iv. employing an algorithm to fit the data produced by said calibrated optical turbidity probe during the course of said first bioprocess run to the analytical model of cell number density N at time (t) wherein (t) denotes a time during the first growth run according to the formula:
$$N ( t ) = {N}_{2} + \frac{{N}_{1} - {N}_{2}}{1 + {e}^{\frac{t - {t}_{0}}{T}}}$$
in order to determine four parameters, of the run, the time constant T, the transition time when the cells move from the exponential growth phase to the linear growth phase wherein t denotes a time during the process according to, the cell density at inoculation N₁, and the maximum cell density carrying capacity for the process N₂;
v. initiating a subsequent bio-process growth run by inoculating a growth medium substantially the same as that utilized in said first growth run with the same cell line as was utilized in said first bio-process growth run
vi. measuring the initial viable cell fraction (V₀) present in the bioreactor growth medium at the time of initiating said subsequent bio-process growth run;
vii. determining the parameters for the cell viability curve in accordance with the equation
$$V ( t ) = {V}_{0} - {V}_{1} {e}^{\frac{t - {t}_{K}}{{T}_{V}}}$$
from the cell growth curve parameters, wherein T_{V}˜T and t_{K}˜t₀+2.71 T, and V₁ is as determined in step iii;
viii. calculating the percentage of viable cells present in said subsequent bio-process growth run at least once during the course of said subsequent bio-process growth run using the parameters determined in step vi), in conjunction with V₀ as measured in step v); and
ix. initiating a change in the bio-process conditions as soon as the percentage of viable cells reaches a pre-determined value, based on the calculation of step viii).

10. The process of claim 9, where the biological, physical and/or chemical properties of the medium are changed as soon as the percentage of viable cells reaches a pre-determined value.

11. The process of claim 10, wherein the temperature and/or pH of the medium is changed as soon as the percentage of viable cells reaches a pre-determined value.

12. The process in claim 10, wherein promoters of apoptosis and cell lysis are added to the bioreactor as soon as the percentage of viable cells reaches a pre-determined value.

13. The process in claim 9, wherein the cells are harvested as soon as the percentage of viable cells reaches a pre-determined value.

14. The process in claim 9, wherein the cells are transfected with an adenovirus or baculovirus as soon as the percentage of viable cells reaches a pre-determined value.

15. A process for determining changes in the instantaneous specific growth rate of cells in a bio-process comprising the steps of:
i) inoculating a growth medium contained in a bio-reactor vessel with cells;
ii) plotting a first curve using a calibrated optical turbidity probe, which first curve plots the number density of said inoculated cells vs. time;
iii) smoothing the data from said first curve using a Savitzky Golay smoothing algorithm;
iv) calculating the first derivative of the smoothed curve to thereby provide a second curve indicative of the instantaneous specific growth rate of said cells relative to the time elapsed since inoculation;
iv) determining any discontinuities in said second curve; and
vi) recording the time at which said discontinuities occur relative to the time elapsed since said inoculation; and

16. A process in accordance with claim 15 wherein the temperature and/or pH of the medium is changed on the occurrence of a discontinuity.

17. A process in accordance with claim 15 wherein promotors of apoptosis and cell lysis are added to the growth medium on the occurrence of a discontinuity.

18. A process for determining changes in the instantaneous specific growth rate of cells in a bio-process comprising the steps of:
i) inoculating a growth medium contained in a bio-reactor vessel with cells;
ii) plotting a first curve using a calibrated optical turbidity probe, which first curve plots the number density of said inoculated cells vs. time;
iii) smoothing the data from said first curve by employing a Savitzky Golay smoothing algorithm;
iv) calculating the first derivative of the smoothed curve to thereby provide a second curve indicative of the instantaneous specific growth rate of said cells relative to the time elapsed since inoculation; iv) determining from said subsequent curve when the specific growth rate decreases to substantially zero.

19. A process in accordance with claim 18 wherein said cells are harvested when said specific growth rate decreases to substantially zero.
